(19) United States
(12) Patent Application Publication
(10) Pub. No.: US 2008/0070993 A1
(43) Pub. Date: Mar. 20, 2008

(54) Additives for cosmetic products and the like

(76) Inventor: Janos Borbely, Debrecen (HU)

Correspondence Address: Thomas A. O'Rourke;Bodner & O'Rourke, 425 Broadhollow Road, Melville, NY, 11747, US

(21) Appl. No.: 11/899,608

(22) Filed: Sep. 6, 2007

Related U.S. Application Data
(60) Provisional application No. 60/843,013, filed on Sep. 7, 2006.

(51) Int. Cl. A61K 47/12; A61K 47/36

(52) U.S. Cl. 514/777; 514/784

(57) ABSTRACT

The present invention provides additives for cosmetic compositions. The additives chitosan based compositions, hyaluronic acid based compositions and/or poly Y glutomic acid based compounds. The cosmetic composition can be used in a variety of cosmetic properties.

This application claims priority on U.S. Provisional Patent Application Ser. No. 60/843,013, filed on Sep. 7, 2006, the disclosures of which are incorporated herein by reference.

### FIELD OF THE INVENTION

The present invention is directed to improvements in the field of cosmetic and fragrance products.

### BACKGROUND OF THE INVENTION

Cosmetics are a popular item among many consumers. There are a wide variety of cosmetic products available on the market. These products include lip products such as lip gloss, lipstick, lip balm, etc. There are skin care products that include powders, lotions, emollients, etc. Other cosmetic products include eye shadow, mascara, nail products, etc. There are also hair care products such as shampoo, rinses, conditioners, etc.

Cosmetics are frequently used to alter or modify one's appearance. Traditionally, natural ingredients were used in many cosmetic materials. As time has gone on, many synthetic materials have been used instead.

Related to cosmetics are fragrances such as perfume and the like. The fragrances can be applied as a liquid, solid, semi-solid, paste or other manner.

Many of the cosmetic products currently available contain a base material and one or more additives to facilitate the use of the product, the storage of the products or for other reasons.

### OBJECTS OF THE INVENTION

It is an object of the present invention to provide improved additives for cosmetic products and the like.

### SUMMARY OF THE INVENTION

The present invention relates to cosmetic compositions that include core-shell polymers of chitosan, especially to nano-sized derivatives. Precrosslinked chitosan compounds are prepared by chemical modification of chitosan linear polyamine with natural mono-, di-, tri- and polycarboxylic acids. This core could be hydrophilic, hydrophobic or amphiphilic depending on the ratio of crosslinking and the character of carboxylic acid. In the preferred embodiment, the chitosan first reacts with a carboxyl compound forming a partially crosslinked or modified nanoparticles. The surface of the chitosan compound so formed with a plurality of molecules of shell. More particularly, after amidizing the chitosan, the next step is the reaction of these precrosslinked chitosan compounds with one or more functional carboxylic acid. Functional groups of these molecules can be vinyl groups, and their polymerization by chemical or by UV or blue light initiation can be obtained the hydrophobic shell of nanoparticles or can be stable ions, which can be formed the shell as polyion applying in a complexation with gene DNA or RNA molecules.

Other cosmetic compositions can include as an additive an Hyaluronic acid (HA). Hyaluronic acid (HA) is a linear polysaccharide of alternating disaccharide units of D-glucuronic acid and N-acetyl-D-glucosamine. It is a natural polysaccharide found in a vitreous body, extracellular matrix, connective tissues, synovial fluid, and organs. The molecular weight of the natural biopolymer is in the range of 10 kDa and 6000 kDa. Role of HA is important in the structure and organization of the extracellular matrix, transport of nutrients, cell adhesion and regulation of inflammation. A variety of studies have focused on creating of crosslinked hyaluronan systems. The covalently crosslinking modification commonly result in gel or film formation.

The present inventions includes nanoparticles based on HA by covalently crosslinking via carboxylic groups of the HA chain with a diamino compound in aqueous media at room temperature performed at room temperature. The condensation reaction of amino groups and pendant carboxylic groups of HA was performed by using water soluble carbodiimide. The prepared nanosystems form clear solutions in aqueous media. The structure of products was determined by NMR and IR spectroscopy, the particle size was identified by Laser Light. Scattering (DLS) and Transmission Electron Microscopy (TEM) measurements. It was found, that particle size depends on the ratio of crosslinking and the molecular weight of HA. The crosslinked HA nanoparticles might have role in a wide range of biomedical application, sucs as in fabrication of medical devices and in drug delivery systems.

Another additive that may be used in cosmetic applications includes hydrogels made from poly-.gamma.-glutamic acid (PGA) compounds. Precross-linked poly-.gamma.-glutamic acid (PGA) compounds are prepared by amidizing PGA with amine groups. In the preferred embodiment, the PGA first reacts with a diamino or polyamine compound forming a partially crosslinked nanoparticle. The surface of the PGA compound so formed is provided with a plurality of vinyl groups. Then the vinyl group undergoes radical polymerization forming hydrogel. More particularly, after amidizing the poly-g-glutamic acid, the next step is the reaction of these precross-linked PGA compounds with one or more vinyl groups, and their polymerization by chemical initiation or polymerization preferably through the use of photopolymerization, i.e., upon exposure to light of a predetermined, specified wavelength, to form hydrogels. The preferred wavelength of the light is blue light. The preferred exposure is from about 1 minute to about 2.5 hours. Where chemical initiation is performed instead of photopolymerization, the preferred initiators are potassium-persulphate or ammonium-persulphate, although others may be used as well. As a catalyst in the chemical initiation, TEMED (tetramethyl-ethylene-diamine), or DMAPN (3-dimethylamino-prophyonitrile) are preferred.

### DETAILED DESCRIPTION OF THE INVENTION

The present invention encompasses compositions comprising cosmetically or dermatologically acceptable formulations which are suitable for contact with living animal tissue, including human tissue, with virtually no adverse physiological effect to the user. Compositions embraced by this invention can be provided in any cosmetically and/or dermatologically suitable form, preferably as a solid, semisolid, powder, lotion or cream, but also in an anhydrous or aqueous base, as well as in a sprayable liquid form. Other suitable cosmetic product forms for the compositions of this invention include, for example, an emulsion, a lip balm, a lip gloss, a lotion, a mask, an ointment, a mousse, a patch, a pomade, a solution, a spray, a wax-based stick, or a towelette. In addition, the compositions contemplated by this invention can include one or more compatible cosmetically acceptable adjuvants commonly used and known by the skilled practitioner, such as colorants, fragrances, emollients, humectants, preservatives, vitamins, chelators, thickeners, and the like, as well as other botanicals such as aloe, chamomile, and the like, and as further described below.

In the cosmetic field, lipstick, lip-balm, foundation, pencil shaped cosmetics, and hair stick are known as solid cosmetic compositions. Among these, oil-based solid cosmetic compositions is usually (a) a liquid oil component such as caster oil, jojoba oil, squalene, lanolin, chemically synthesized ester oil, liquid paraffin, and (b) a wax component which is a solidifier such as carnauba wax, candelilla wax, ceresin wax, microcrystalline wax, hydrogenated animal oil, hydrogenated vegetable oil, beeswax, and (c) a powder component. The powder component is dispersed in the mixture of liquid oil component and wax component.

Generally, the product must satisfy the shape-retaining ability and usability. Usability requires especially good spreadability and gloss, which is controlled by the blending ratio of a wax component and a liquid oil component. But it is quite difficult to satisfy the demands for enough shape-retaining ability and good usability simultaneously. Because the wax component is an essential ingredient for solidifying the liquid oil component, it should be a large amount of the formula in order to retain the product shape while in use and in order to allow storing at various temperatures. When the amount of the wax component is high, spreadability of the product tends to be heavy, and the gloss that is provided by the liquid oil component tends to be decreased. Because of this contradiction, it is difficult to satisfy the demands for enough shape-retaining ability and good usability simultaneously. It is also demanded that a solid cosmetic composition have superior shape-retaining ability and usability for a long period of time.

In order to solve the above-mentioned problem, a small amount of wax giving high hardness to the product may be selected Such a wax usually has a high melting point and there is a problem that it is not easy to produce. Such a wax requires additional heating process in order to melt the wax ingredient at a high temperature during production and the filling property tends to become undesirable due to the relatively high melting point. Further more, when the solid cosmetic composition contains water such as an emulsified type lipstick, extra heating at high temperature causes loss of the water ingredient.

The invention applies not only to make-up products for the lips, such as lipsticks, lip glosses and lip pencils, but also to care and/or treatment products for the skin, including the scalp, and for the lips, such as antisun products especially in stick form for the skin of the face, the body or the lips, make-up products for the skin, both of the human face and of the human body, such as foundations optionally cast in stick or dish form, concealer products, eye shadows, and transfer tattoos, body hygiene products such as deodorants, especially in stick form, shampoos, conditioners and make-up products for the eyes such as eyeliners, eye pencils and mascaras, more especially in cake form, as well as care products for the face, the body and keratin fibres such as the hair and the eyebrows. The composition of the invention can be in the form of a paste, a solid or a more or less viscous cream. It can be an oil-in-water or water-in-oil emulsion, or a rigid or soft anhydrous gel. In particular, it is in a form cast as a stick or a dish and more especially in the form of an anhydrous rigid gel, especially an anhydrous stick. More especially, it is in the form of a rigid gel that is translucent or transparent, the liquid fatty phase forming the continuous phase. The gelling of the oil can be modified according to the nature of the hetero atom-containing polymer used, and may be such that a rigid structure in the form of a tube or a stick is obtained. When these tubes are coloured, they make it possible, after application, to obtain a glossy deposit of uniform colour, that does not transfer, in particular onto a support placed in contact with the film, after evaporation of the volatile solvent, and that has good staying power, especially of the color over time.

One type of additive that may be included in the cosmetic composition of the present invention is a chitosan based material.

Chitosan Based Compositions

The starting material of the present invention is chitosan as a derivative of natural chitin. After cellulose, chitin is the second most abundant natural biopolymer on earth. One of the derivatives of chitin is chitosan, obtained by N-deacetylation. Chitosan is a copolymer of N-acetylglucosamine and glucosamine, biocompatible and biodegradable linear polyamine. A limiting factor in the modification and application of chitosan is its low solubility. Chitosan is soluble in aqueous acidic media and forms viscous solution. Chitosan and its solution have found wide variety of applications, such as medicine, pharmaceutics, food technology.

Chitosan was purified by dissolution in hydrochloric acid and by precipitation with NaOH. The purified chitosan was washed with distilled water and freeze dried.

After freeze drying, chitosan is then partially amidated by reacting it with di-, tri- or polycarboxylic acid compounds. Preferred dicarboxyl compound are:

Aliphatic di/tri/polycarboxylic acid, such as:
HOOC—(CH₂)ₙ—COOH n=0 to 4
Oxalic acid
Malonic acid
Succinic acid
Glutaric acid
Adipic acid
Hydroxi-di/tri/polycarboxylic acids, such as
HOOC—(CH₂)ₙ—(CHOH)ₘ—COOH n=0 to 2 and m=0 to 2, such as:

Malic acid

Tartaric acid

Citric acid

2-hydroxi-maleic acid

1,3-acetonedicarboxylic acid

Mucic acid

Oxo-di/tri/polycarboxylic acid, such as:

Oxalacetic acid

Aromatic di/tri/polycarboxylic acids, such as:

Phthalic acid

Terephthalic acid

Unsaturated di/tri/polycarboxylic acid, such as:

Maleic acid

Fumaric acid

Muconic acid

Others, such as:

Poly-(ethyleneglycol)-bis-(carboxymethylether)

The reaction that is performed determinates the precrosslinking of chitosan. This precrosslinking can performed so that there different amounts of crosslinking in the final product, i.e., from 1 to 99% crosslinking. The amidizing reaction takes place in water, in the presence of a water soluble carbodiimide (CDI) compound, which preferably is 1-[3-(dimethylamino)propyl]-3-ethylcarbodiimide hydrochloride.

The core is formed by crosslinking the chitosan or by self-assemble of chitosan linear chain. The partial precrosslinked products can be further vinylized with different compounds If desired. The content of vinyl groups is preferably from about 5 to about 50%, more preferably 10 to 30%, reported to the free amino groups from the precrosslinked chitosan products. Preferred water soluble carboxylic acid containing vinyl groups are such as:

Acrylic acid

Methacrylic acid

In this second stage the outer shell is formed from hydrophobic compounds that optionally may be crosslinked a priori. The core-shell morphology may be a result of self assemble of modified chitosan in the case when hydrophobic side chains are introduced. Therefore, in aqueous solution the hydrophobic chains are in the inner part and the residual hydrophilic segments occupy the corona. These particles are design for solubilization of water insoluble lipophilic compounds or drugs. Preferred monocarboxylic acid are:

Aliphatic monocarboxylic acid, such as:

n-butyric acid

Propionic acid

Hydroxi-monocarboxylic acids, such as

Salicylic acid

Mandelic acid

Aromatic monocarboxylic acids, such as:

Benzoic acid

Phenylacetic acid

Heteroaromatic monocarboxylic acid, such as:

Nikotinic acid

The partial precrosslinked chitosan can be further amidated with different compounds If desired to obtain core-shell morphology. The content of functional groups is preferably from about 5 to about 50%, more preferably 10 to 30%, reported to the free amino groups from the precrosslinked chitosan products. Preferred carboxylic acids have high hydrophilic character or stable charge to form hydrophilic and/or charged shell. Preferred carboxylic acid, such as:

Poly-(ethyleneglycol)-carboxylic acid

Betaine hydrochloride

The core and the outer shell can be formed hydrophobic or hydrophilic. The reaction takes place in a multi-step process. The formation of the primary crosslinked core is followed by formation of a covalently attached shell. The reaction is controlled by the conditions of reaction (e.g., concentration, molar ratio) to obtain a very slightly crosslinked core wherein the porosity is very high, or to obtain a very highly crosslinked core wherein the porosity is very low. The degree of crosslinking could be in the range of from about 1% to about 99%.

Hyaluronic Acid (HA) Based Compositions

Other cosmetic compositions can include as an additive an Hyaluronic acid (HA). Hyaluronic acid (HA) is a linear polysaccharide of alternating disaccharide units of D-glucuronic acid and N-acetyl-D-glucosamine. It is a natural polysaccharide found in a vitreous body, extracellular matrix, connective tissues, synovial fluid, and organs. The molecular weight of the natural biopolymer is in the range of 10 kDa and 6000 kDa. Role of HA is important in the structure and organization of the extracellular matrix, transport of nutrients, cell adhesion and regulation of inflammation. A variety of studies have focused on creating of crosslinked hyaluronan systems. The covalently crosslinking modification commonly result in gel or film formation.

The present inventions includes nanoparticles based on HA by covalently crosslinking via carboxylic groups of the HA chain with a diamino compound in aqueous media at room temperature performed at room temperature. The condensation reaction of amino groups and pendant carboxylic groups of HA was performed by using water soluble carbodiimide. The prepared nanosystems form clear solutions in aqueous media. The structure of products was determined by NMR and IR spectroscopy, the particle size was identified by Laser Light Scattering (DLS) and Transmission Electron Microscopy (TEM) measurements. It was found, that particle size depends on the ratio of crosslinking and the molecular weight of HA. The crosslinked HA nanoparticles might have role in a wide range of biomedical application, such as in fabrication of medical devices and in drug delivery systems.

Hydrogels Made from Poly-γ-Glutamic Acid (PGA) Compounds

The starting material of the additive of the present invention is PGA which is prepared by fermentation with a suitable microorganism, capable of producing PGA in a suitable fermentation medium, under conditions and time appropriate for the microorganism used. The resulting culture medium is treated, by centrifugation, to separate the cells from the PGA. The resulting cell-free liquid is treated with acetone to obtain the PGA from this fermentation medium. After obtaining the PGA from the fermentation medium, the PGA so obtained was purified by dialysis and subsequently freeze dried. The molecular weight of the PGA is typically about 1,000,000.

After freeze drying, the PGA is then partially amidated by reacting it with a diamino or polyamino compound. A preferred diamino compound is a linear di/tri/polyamines. Other preferred diamino or polyamino compounds can include heterocyclic di/tri/polyamines, such as piperazine, aromatic di/tri/polyamines, such as 1,4-diphenyl amine, and heteroaromatic di/tri/polyamines, such as adenine. Other diamino or polyamino compounds can include one or more of the following or blends thereof:

1,3-diaminoacetone

2,4-diaminobutyric acid

1,3-diaminoguanidine

1,3-diamino-2-propanol

Cycloaliphatic di/tri/polyamine such as:

1,8-diamino-p-menthane

2,5-diazabicyclo[2.2.1]heptane

Heterocyclic di/tri and polyamine, such as:

piperazine-2-carboxylic acid

Aromatic di/tri/polyamine, such as:

2,5-diaminobenzenesulfonic acid

3,5-diaminobenzoic acid

Heteroaromatic di/tri/polyamine, such as:

2,6-diaminopurine

2,3-diaminopyridine

2,5-diaminopyridine

2,6-diaminopyridine

The amidizing reaction that is performed determinates the precross-linking of the PGA. This precross-linking can performed so that there different amounts of crosslinking in the final product, i.e., from 1 to 99% cross linking. The amidizing reaction takes place in water, in the presence of a water soluble diamide compound, which preferably is dimethylamino propyl ethylcarbodiimide methiodide.

These partial precross-linked products can be further vinylized with different compounds If desired. These vinylizing compound include but are not limited to AEM (aminoethyl methacrylate hydrochloride) and other water soluble vinyl monomers containing amino functionality. The content of vinyl groups is preferably from about 5 to about 50%, more preferably 10 to 30%, reported to the free carboxyl groups from the precross-linked PGA products. Using these products (precross-linked and vinylized PGA) IPN hydrogels were obtained, using other soluble monomers, including but not limited to any one of the following or blends thereof:

acrylic acid

acrylic anhydride

acrylic acid anhydride

2-acrylamino-2-methyl-1-propanesulfonic acid

2-acryloxyetheyltrimethylammonium chloride

N-acryloxysuccinimide

Bis(2-acryloxyethyl)phosphate

2-carboxyethyl acrylate

glycerol monoacrylate

hydroxyethyl acrylate

hydroxypropyl acrylate

itaconic acid

monoacryloxyethyl phosphate

methacylic acid

methacrylic acid anhydride

cinnamyl methacrylate

glycerol monomethacrylate

hydroxyethyl methacrylate

hydroxypropyl methacrylate

methacryloyltris(hydroxymethyl)methylamine

N-methyl-N-vinylacetamide

poly(ethyleneglycol)di/monomethacrylate

poly(propyleneglycol)di/monomethacrylate

N-vinyl-2-pyrrolidone

1-vinylimidazole

vinylsulphonic acid

N-vinyl urea

The pH and ionic strength of the final hydrogel can be readily adjusted by changing the acidic, basic or neutral functions. Alternatively, the formation of the hydrogels may be accomplished through the use of polymers which are water soluble but which do not react with the vinylized cross-linked PGA, and instead just penetrate its polymer network forming a SEMI-IPN (semi-interpenetrating polymer network). The polymers which can be used to form a SEMI-IPN include but are not limited to the following:

Natural: polylisine, polyasparagine, chitosan, alginates, hyaluronic acid

Synthetic: polyacrylic/methacylic acid, poly-N-vinyl pyrolidone

The additives of the present invention may be used in a variety of cosmetic, hair and skin products including but not limited to any of the following. The additives may be present in an amount of 0.001 to about 5% by weight. In other embodiments, the upper range of the additive may be in the range of 5% to about 25% or more.

Lipsticks and Pomades

The lipstick or pomade of the present invention typically includes one or more base material that is generally about 30.00% to about 95.00% or more by weight of the total weight of the composition, colorants are up to about 15.00% or more by weight, fragrances up to about 3.00% by weight, and other ingredients, such as skin protectants, are about 0.01% to about 20.0% by weight. The skin protectants may include allantoin in an amount about 0.50% to about 2.0% by weight. The carrier or base preferably comprises about 60% to about 90% and, most preferably, about 70% to 80%% by weight of the total weight of the lip composition, and includes moisturizers, which may make up part or most of the carrier. Preferred moisturizers include lanolin, vegetable oil, castor oil, isopropyl palmitate, mineral oil, petrolatum, avocado oil, soybean oil, caprylic/capric triglycerides, diisopropyl dimerate, methicone or a combination thereof. The lip composition of the present invention may also includes up to about 1%, more preferably up to about 0.60% of a healing agent. Preferred healing agents include vitamin E, allantoin, candelilla wax, aloe vera, petrolatum, calamine, dimethicone, cocoa butter, shark liver oil, glycerin, zinc oxide, aluminum hydroxide, kaolin, zinc acetate, zinc carbonate or a combination thereof. The lip composition may also include an occlusive wax. Preferred waxes include candelilla wax, ceresine wax, ozokerite, lanolin wax, beeswax, paraffin, microcrystalline wax, animal wax, plant wax, synthetic wax, mineral wax, Japan wax, spermaceti, sunflower wax, orange wax, shellac wax, rice wax, jojoba wax, rose wax, jasmine wax or a combination thereof. Additionally, certain lip compositions may include a colorant or additional active ingredients. Preferred active ingredients include allantoin, aloe vera, elastin, collagen, vitamin E and derivatives thereof, vitamin A and derivatives thereof, liposomes, sodium hyaluronate, water, botanical extracts, phospholipids, silk powder, evening primrose oil, cholesteryl blo glutamate, hyaluronic acid, rose hips oil, lauroyl lysine or a combination thereof.

Lip Balms

The compositions of the present invention may also be used in lip balms. Generally, the lip balm of the present invention may be in any suitable form such as a stick, semisoft material, liquid etc. Lip balm compositions for topical application may comprise a wax or other pharmaceutically acceptable vehicle and, optionally, one or more medicaments and/or adjuvants. Suitable waxes include, but are not limited to, petrolatum wax; carnauba wax; paraffin wax; white wax; candelilla wax; beeswax; oils, such as arachidyl propionate, cetyl alcohol, isopropyl lanolate, isopropyl myristate, lanolin, mineral oil, light mineral oil, octyldodecanol, oleyl alcohol, ethyl macadamiate, castor oil, jojoba esters, hydrogenated castor oil, hydrogenated vegetable oil, cetyl ricinoleate, propylene glycol, isopropyl palmitate, stearyl alcohol, and volatile and non-volatile silicone oils; and any combination of any of the foregoing. Suitable silicone oils include, but are not limited to, polyphenylmethyl siloxane, dimethicone, cyclomethicone, and any combination of any of the foregoing.

Suitable minor components include, but are not limited to, sunscreens, such as octyl methoxycinnamate, octyl dimethyl p-aminobenzoate, actinoquinol, p-aminobenzoic acid, butyl methoxydibenzoylmethane, beta-carotene, 4-dimethylamino benzoic acid, dioxybenzone, drometrizole, lawsone, sulisobenzone, titanium dioxide, and zinc oxide; preservatives, such as parabens, including but not limited to, methylparaben, isopropylparaben, and isobutylparaben; flavorants; fragrances; colorants, such as beta-carotene; conditioners, such as skin conditioning agents, including but not limited to, the aforementioned waxes and oils, especially petrolatum, dimethicone, and polymethylsiloxane; moisturizers; emollients; cleansing agents; antioxidants, such as tocopherol; antistatic agents, such as cocamidopropyl PG-dimonium chloride; stabilizers; and pH adjusters, such as sodium hydroxide and hydrochloric acid. Preferably, each such component is approved for use in or on humans by the Food and Drug Administration, or corresponding regulatory bodies in other regions or countries.

Liquid Lip Products

The present invention may also include liquid lip products. The liquid lip product or liquid lipstick of the present invention in a broad sense includes as its principal ingredients a film forming polymer, a pigment, an alcoholic solvent, and one or more thickeners, humectants, moisturizers, and the like. Further, the liquid lipstick may advantageously contain natural flavors and herbs to improve the flavor, appearance, sensation on the lips, and dermatologic properties of the product.

These liquid lip products may include about 50% to about 90% of an alcoholic solvent; about 2% to about 20% of a vinyl acetate copolymer; about 0.10% to about 5% of a non-toxic glycol; about 0.09% to about 10% of cyclomethicone and aluminum magnesium hydroxide stearate; about 1% to about 7% decamethyl cyclopentasiloxane and trimethylsilicate; and about 0.01% to about 5% of a dye or pigment. Preferably, the liquid lipstick also includes natural herbs and other ingredients such as about 0.001% to about 1.00% of chamomile extract; about 0.01% to about 1.00% of jojoba oil; about 0.01% to about 1.00% of allantoin; about 0.01% to about 1.00% of aloe vera gel; and about 0.01% to about 1.00% of octylmethoxycinnamate. The composition of the present invention advantageously includes lubricants such as silicone polymers. Preferably, a cosmetic formulation product including decamethyl cyclopentasiloxane (and) trimethylsiloxysilicate. The lipstick may include additional lubricants, such as the natural ingredient jojoba oil. Other useful lubricants include silicone or mineral oil.

As a film forming polymer, a number of polymers may be used. Preferably, the polymer is Resyn 28-2930, a vinyl acetate/crotonates/vinyl neodecanoate copolymer, but such polymers as Amphomer, Dermacryl, Carboset, Lovocryl, among other polymers may also be used. The amount of polymer used varies on the particular properties of the polymer selected, but if the polymer is Resyn 28-2930, for example, as is preferred, approximately 2 to 20% can be used.

The solvent for the foregoing plasticizer or film former polymer should preferably be an alcohol approved for use in formulating cosmetics, such as denatured alcohol (ethanol), in the form of SDA-40 denatured alcohol. It can be used in an amount varying from approximately 40% to about 90% or more and more preferably from 60% to 80%. Other organic solvents which may be employed in addition to, or instead of denatured alcohol, such as stearyl alcohol, isostearyl alcohol, and fatty acids which are water-insoluble, such as lauric acid, stearic acid, myristic acid, palmitic acid, and lanolin fatty acid. Lanolin may also be used in the organic solvent.

The pigments and dyes used in the formulations in the present invention include standard pigments used in the industry, and identified by FD&C designations and D&C designations, but other listed and approved natural colors compatible with alcoholic solvents can be used. In addition, any non-toxic pigment certified by FD&C or D&C can be used. Preferably, a combination of pigments may be used to obtain the desired lipstick color. Such commonly used pigments as FD&C Yellow No. 5 (C69-002), Red No. 7 (C19-011), Red No. 22 (C14-6634), and Red No. 21 (C14-032) may be used in amounts for each pigment from about 0.01% to about 6%, to obtain a desired red color, for example. Where alcohol-soluble pigments are used, dispersion in caster oil becomes unnecessary.

Others additives and ingredients enhance the liquid lipsticks of the present invention, including one or more humectants, viscosity builders, lubricants, UV protectors, herbs, and flavors. The humectant may be propylene glycol, or another glycol or glycol polymer or sorbotol or glycerin. Preferably, the humectant is propylene glycol, and it should be present in an amount from about 0.10% to about 5%, with 1 to 2% more preferred. The viscosity builder may include a mixture of Cyclomethicone (and) Aluminum Magnesium Hydroxide Stearate in an amount ranging from about 1% about 7%, or more preferably about 2.5% to about 4%. The viscosity builder or thickener may include other silicone polymers available from GE Silicones including cyclomethicone, dimethicone and mixtures thereof, or other GE silicone polymers suitable for personal use. Alternatively, lanolin alcohol or hydrogenated castor oil may be used.

Sunscreens

The sunscreen containing compositions of the present invention will include a sunscreen, such as octyl methoxycinnamate, octyl dimethyl p-aminobenzoate, actinoquinol, p-aminobenzoic acid, butyl methoxydibenzoylmethane, beta-carotene, 4-dimethylamino benzoic acid, oxybenzone, dioxybenzone, drometrizole, lawsone, sulisobenzene, titanium dioxide, zinc oxide, and any combination of any of the foregoing.

Deodorants

Typical compounds (high molecular weight hydrocarbons, alcohols, and acids) included in deodorant compositions of the present invention can include, but are not limited to, stearic acid, stearyl alcohol, propylene glycol, cyclomethicone, and any combination of any of the foregoing. The deodorant compositions will also include one or more deodorants, such as (though not limited to) cocamidopropyl PG-dimonium chloride phosphate, abietic acid, aluminum citrate, aluminum PCA, azadirachta indicia extract, chlorophyllin-copper complex, eugenia jambolana extract, farnesol, fermented vegetable extract, ginger lily (Hedychium spicatum) extract, .alpha.-glucan-oligosaccharide, mauritia flexosa extract, octoxyglycerin, salvia miltiorrhiza extract, sandalwood (Santalum album) extract, sodium aluminum chlorohydroxy lactate, spondias amara extract, triethyl citrate, zinc phenolsulfonate, zinc ricinoleate, and any combination of any of the foregoing. They may also, or alternatively, contain one or more antiperspirants, including, but not limited to, allantoin-aluminum chlorohydrate, aluminum capryloyl hydrolyzed collagen, aluminum chlorhydrex GLY, aluminum chloride, aluminum chlorohydrate, aluminum chlorohydrex, aluminum PCA, aluminum sesquichlorohydrate, aluminum undecylenoyl collagen amino acids, aluminum zirconium pentachlorohydrate, aluminum zirconium tetrachlorohydrate, aluminum zirconium tetrachlorohydrex GLY, aluminum zirconium trichlorohydrate, aluminum zirconium pentachlorohydrate, sage (Salvia officinalis) extract, tormentil (Potentilla erecta) extract, zirconium chlorohydrate, and any combination of any of the foregoing. Preferred minor components of the deodorant composition include a medicament; colorant; fragrance; and conditioner.

Skin Cream

The compositions of the present invention yield improvements to the aesthetic appearance of the skin by treating at least one of the following: dermatological aging, especially chronological, actinic or hormonal aging. In particular, improvements to the aesthetic appearance of skin include at least one of the following: makes facial lines appear less noticeable, makes facial lines and/or wrinkles feel plumped, improves appearance of suborbital lines and/or periorbital lines, improves appearance of crow's feet, reduces and/or diminishes the appearance of wrinkles, particularly facial wrinkles on the cheeks, forehead (e.g. perpendicular wrinkles between the eyes, horizontal wrinkles above the eyes) and/or around the mouth (e.g. marionette lines), and particularly deep wrinkles or creases, rejuvenates and/or revitalizes skin, particularly aging skin, decreasing hyperkinetic facial wrinkling and reduces and/or eliminates fine and/or deep lines. Also, embraced by the present invention are transdermal modes of delivery, such as patches and the like, with or without a suitable penetration enhancers. The methods and compositions embodied by the invention provide a means by which the components can be effectively administered in a transdermal system. Accordingly, a transdermal means of delivering a composition or formulation (often with a penetration enhancing composition) to the skin is that of the transdermal patch or a similar device as known and described in the art. The transdermal mode of storing and delivering the compositions onto the skin and forming the active composition is convenient and well suited for the purposes of an embodiment of the present invention. In a preferred method, the application is through a sustained release vehicle, e.g., a topically applied sustained released patch. Preferably, when a topical patch is used, the patch is applied to the desired area for extended period of time. Preferably, the extended period of time is greater than one hour, most preferably the extended period of time is overnight, i.e., when the user is sleeping. Another particular embodiment of the present invention is directed to the delivery of the described compositions by the use of targeted delivery systems, for example, liposomes, microspheres (see, e.g., U.S. Pat. No. 5,770,222 to Unger et al.), and the like, so that the components and/or active constituents can more readily reach and affect the muscle layer of the area of application, e.g., face or neck, or the other area of the skin. In another preferred embodiment, the topical compositions of the present-invention also include at least one of the following: a skin penetration enhancer, a surface smoother, a skin plumper, an optical diffuser, a sunscreen, an exfoliation promoter, and an antioxidant. A surface smoother provides the functional benefits of enhancing skin smoothness and reducing the appearance of fine lines and coarse wrinkles. Examples include isopropyl myristate, petrolatum, isopropyl lanolate, silicones (e.g., methicone, dimethicone), or any mixtures thereof. The surface smoother is preferably present from about 0.1 wt % to about 50 wt % of the total weight of the composition. A skin plumper serves as a collagen enhancer to the skin. An example of a suitable, and preferred, skin plumper is palmitoyl oligopeptide. Other skin plumpers are collagen and/or glycosaminoglycan (GAG) enhancing agents. The skin plumper is preferably present from about 0.1 wt % to about 20 wt % of the total weight of the composition. An optical diffuser is a particle that changes the surface optometrics of skin, resulting in a visual blurring and softening of, for example, lines and wrinkles. Examples of optical diffusers that can be used in the present invention include, but are not limited to, boron nitride, mica, nylon, polymethylmethacrylate (PMMA), polyurethane powder, sericite, silica, silicone powder, talc, Teflon, titanium dioxide, zinc oxide, or any mixtures thereof. The optical diffuser is preferably present from about 0.01 wt % to about 20 wt % of the total weight of the composition. A sunscreen protects the skin from damaging ultraviolet rays. In an illustrative embodiment of the present invention, the sunscreen would provide both UVA and UVB protection, by using either a single sunscreen or a combination of sunscreens. Among the sunscreens that can be employed in the present compositions are avobenzone, cinnamic acid derivatives (such as octylmethoxy cinnamate), octyl salicylate, oxybenzone, titanium dioxide, zinc oxide, or any mixtures thereof. The sunscreen may be present from about 1 wt % to about 30 wt % of the total weight of the composition. The addition of a sunscreen may prevent/reduce the photodegradation of the composition while in the package as well as serve to protect the skin from ultraviolet radiation.

The compositions of the present invention may have a having sunscreen bring about additional improvements to the aesthetic appearance of skin, including at least one of the following: minimizes sunburning, minimizes tanning, and reduces redness. The present compositions may also have one or more exfoliation promoters. Suitable examples of an exfoliation promoter that can be used in the present compositions include alpha hydroxy acids (AHA); benzoyl peroxide; beta hydroxy acids; keto acids, such as pyruvic acid, 2-oxopropanoic acid, 2-oxobutanoic acid, and 2-oxopentanoic acid; oxa acids as disclosed in U.S. Pat. Nos. 5,847,003 and 5,834,513 (the disclosures of which are incorporated herein by reference); salicylic acid; urea; or any mixtures thereof. The preferred exfoliation promoters are 3,6,9-trioxaundecanedioic acid, glycolic acid, lactic acid, or any mixtures thereof. When the present invention includes an exfoliation promoter, the composition has about 0.5 wt % to 30 wt %, preferably about 1 wt % to about 15 wt %, more preferably about 4 wt % to about 10 wt %, and most preferably about 4 wt %, of the exfoliation promoter based on the total weight of the composition.

An antioxidant functions, among other things, to scavenge free radicals from skin to protect the skin from environmental aggressors. Examples of antioxidants that may be used in the present compositions include compounds having phenolic hydroxy functions, such as ascorbic acid and its derivatives/esters; beta-carotene; catechins; curcumin; ferulic acid derivatives (e.g. ethyl ferulate, sodium ferulate); gallic acid derivatives (e.g. propyl gallate); lycopene; reductic acid; rosmarinic acid; tannic acid; tetrahydrocurcumin; tocopherol and its derivatives; uric acid; or any mixtures thereof. Other suitable antioxidants are those that have one or more thiol functions (—SH), in either reduced or non-reduced form, such as glutathione, lipoic acid, thioglycolic acid, and other sulfhydryl compounds. The antioxidant may be inorganic, such as bisulfites, metabisulfites, sulfites, or other inorganic salts and acids containing sulfur. Compositions of the present invention may have an antioxidant preferably from about 0.001 wt % to about 10 wt %, and more preferably from about 0.001 wt % to about 5 wt %, of the total weight of the composition.

The present composition may also have one or more of the following active agents, ingredients or adjuvants: anesthetics, anti-allergenics, antifungals, antiseptics, chelating agents, colorants, demulcents, emollients, emulsifiers, fragrances, humectants, lubricants, moisturizers, pH adjusters, pigment altering agents, preservatives, stabilizers, surfactants, thickeners, viscosity modifiers, vitamins, or any mixtures thereof. The amounts of these various substances are those that are conventionally used in the cosmetic or pharmaceutical fields, for example, they can constitute from about 0.01% to 20% of the total weight of the composition. Nonlimiting examples of active agents for formulating into the compositions of the present invention include those reagents having an effect on the treatment of wrinkles and/or fine lines, in addition to the Purslane actives as described, such as keratolytic agents, i.e., an active agent having desquamating, exfoliant, or scrubbing properties, or an active agent which can soften the horny layer of the skin. Other examples of anti-wrinkle or anti-fine line active agents include hydroxy acids and retinoids. These agents can be formulated, for example, in amounts of from about 0.0001% to 5% by weight relative to the total weight of the composition. Suitable hydroxy acids include, for example, glycolic acid, lactic acid, malic acid, tartaric acid, citric acid, 2-hydroxyalkanoic acid, mandelic acid, salicylic acid and alkyl derivatives thereof, including 5-n-octanoylsalicylic acid, 5-n-dodecanoylsalicylic acid, 5-n-decanoylsalicylic acid, 5-n-octylsalicylic acid, 5-n-heptyloxysalicylic acid, 4-n-heptyloxysalicylic acid and 2-hydroxy-3-methylbenzoic acid or alkoxy derivatives thereof, such as 2-hydroxy-3-methyoxybenzoic acid.

Exemplary retinoids include, without limitation, retinoic acid (e.g., all-trans or 13-cis) and derivatives thereof, retinol (Vitamin A) and esters thereof, such as retinol palmitate, retinol acetate and retinol propionate, and salts thereof.

In an embodiment embracing topical application, the compositions of this invention comprise a medium (vehicle, diluent or carrier) that is compatible with human skin. The compositions can be formulated as aqueous, alcohol, or aqueous/alcohol-based solutions, ointments, lotions, gels, water-in-oil, oil-in-water, of water-oil-water triple emulsions having the appearance of a cream or gel, microemulsions, or aerosols. In addition, the compositions can be in the form of vesicular dispersions containing ionic and/or nonionic lipids, as described above. Dosage units suitable for such compositions are formulated according to the conventional knowledge and techniques used in the art.

More particularly, the compositions for topical application can be in the form of a protective care composition for the skin, preferably for the face, the neck, the hands, the feet, or other areas of the body. Nonlimiting examples include day creams or lotions, night creams or lotions, sunscreen creams, lotions, or oils, body milks, makeup (a foundation), artificial tanning compositions, depilatories, and patches. There may be one of more plant components present which are preferably contained in a cosmetically or dermatologically acceptable vehicle, medium, diluent or carrier. Emulsifiers are typically present in the compositions of the invention in an amount of about 0.1% to 30%, by weight and preferably from about 0.5% to 30% by weight relative to the total weight of the composition. However, not all compositions will necessarily include emulsifiers.

Hair Products

As used herein the hair treated may be “living” i.e. on a living body or may be ‘non-living’ i.e. in a wig, hairpiece or other aggregation of non-living fibres, such as those used in textiles and fabrics. Mammalian, preferably human hair is preferred. However wool, fur and other melanin containing fibres are suitable substrates for the compositions according to the present invention. Hair is composed of keratin, a sulfur-containing fibrous protein. The present invention related to various hair care compositions. Shampoo compositions of the present invention comprising various combinations of detersive surfactants and conditioning agents. The primary ingredient in the shampoos of the present invention is water, typically making up about 70-80% of the entire formula. Deionized water, is preferably used in the shampoos. Another ingredient in shampoos of the present invention are the primary detergents. These materials, also known as surfactants, are the cleansing ingredients in shampoos. The chemical nature of a surfactant allows it to surround and trap oily materials from surfaces. One portion of the molecule is oil compatible (soluble) while the other is water soluble. When a shampoo is applied to hair or textiles, the oil soluble portion aligns with the oily materials while the water soluble portion aligns in the water layer. When a number of surfactant molecules line up like this, they form a structure known as a micelle. This micelle has oil trapped in the middle and can be washed away with water, thus giving the shampoo its cleansing power. Some preferred detergents used in shampoos of the present invention include ammonium lauryl sulfate, sodium lauryl sulfate, and sodium lauryl ether sulfate and others.

In addition to cleansing surfactants, other types of surfactants may be added to shampoos to improve the foaming characteristics of the formulation. These materials, typically, alkanolamides, help increase the amount of foam and the size of the bubbles. Preferred materials include lauramide DEA or cocamide DEA. The shampoos of the present invention may also include one or more thickening agents to increase the viscosity of the shampoo. These may include methylcellulose. Sodium chloride (salt) also can be used to increase shampoo thickness. The composition of the present invention may also include one or more conditioning agents. Typical conditioning agents can include silicones, and quaternary agents. Other examples of conditioning agents include guar hydroxypropyltrimonium chloride, dimethicone, and quatemium 80.

Preservatives may also be added to the composition. Two of the most preferred preservatives are DMDM hydantoin and methylparaben. Other ingredients may also be added to the shampoo formula to modify specific characteristics. Opacifiers are added to make the formula opaque and give it a pearly look. Materials known as sequestering agents can be added to offset the dulling effects of hard water. Acids or bases such as citric acid or sodium hydroxide can be added to adjust the pH of a shampoo so the detergents will provide optimal cleaning.

The shampoo may also include one or more fragrance oils and/or FD&C dyes. Natural materials such as botanical extracts, natural oils, proteins, and vitamins all impart special qualities to the shampoo. Other additives are dyes which can color the hair.

Anti-dandruff shampoos of the present invention typically incorporate an anti-dandruff active and detersive surfactants. Among the preferred type of anti-dandruff agents are particulate, crystalline anti-dandruff agents, such as sulfur, selenium disulfide and heavy metal salts of pyridinethione. Soluble anti-dandruff agents, such as ketoconazole, are also known in the art. The anti-dandruff shampoos can also provide conditioning benefits from anionic surfactants, cationic polymers and zinc pyridinethione, as an anti-dandruff agent. Conditioning agents such as silicone fluids can optionally be incorporated into the compositions therein.

Mascara

The invention also comprises a composition for making up the eyes and skin comprising a liquid carrier containing at least one film forming polymer, and dispersed therein at least one, preferably a mixture, of organic or inorganic pigments forming the main color component of the composition. Preferably the composition exists in a single phase, rather than in emulsion (water-in-oil or oil-in-water) form. The term “single phase” means that the composition exists in one homogeneous phase (such as an oil phase) and the organic pigments used in the composition are dispersed in that phase and the film forming polymer is solubilized in that phase. The term “single phase” also means that one or more of the film forming polymers and the organic pigments may be dispersed in the liquid vehicle and both are compatible and stable therein. In the latter situation, while the film forming polymer may not be completely soluble in the liquid vehicle, it is capable of dispersing in sufficiently small particles throughout the liquid vehicle, remaining dispersed therein in a stable manner, and compatible therewith. In either case, when the composition is applied to the desired surface, the liquid carrier evaporates at least in part and the film forming polymer sets on the surface trapping the pigment particles that were dispersed in that phase on the surface. Preferably, the composition of the invention has a viscosity ranging from 1000 to 500,000, more preferably 5000 to 250,000, most preferably 7000 to 120,000 centipoise at 25.degree. C. The organic pigments used in the claimed compositions comprise the main color component of the composition. The term “main color component” means that the pigments are present in an amount sufficient to provide color to the composition, meaning that if the amount of organic pigment which is present is removed the color of the composition will be different when a sample of that composition is drawn across the back of the hand, for example, and visually observed with the naked eye. Preferably, organic pigments provide at least about 0.1-95%, preferably at least about 45-80%, most preferably at least about 80-90% of the color of the claimed composition. In the most preferred embodiment, the organic pigments provide about 100% of the color of the claimed composition (meaning that when the entire pigment concentration is measured, the percentage of organic pigments in the entire pigment load is reflected in the percentage mentioned). It is possible that the claimed compositions may contain one or more inorganic pigments including but not limited to metal oxides such as titanium, iron, oxides such as black, red, yellow, green, and blue, and similar organic powders.

The compositions of the invention may be in the liquid, solid, or semi-solid form. Preferably, the compositions are liquids or semi-solids.

I. Pigments and Particulate Fillers

A. Organic Pigments

The composition of the invention preferably comprises about 0.05-30%, preferably about 0.1-25%, more preferably about 0.5-20% by weight of the total composition of one or more organic pigments or salts thereof. The organic pigments should be dispersible in the liquid carrier. Particularly preferred are organic pigments that are red, green, blue, yellow, violet, orange, and mixtures thereof. Also suitable are Lakes of such pigments, which means that the organic pigments are reacted with a metal salt such as calcium, aluminum, barium, zirconium, and the like to form salts. Particularly preferred are aluminum Lakes of the organic pigments, which is where the organic pigment is reacted with aluminum to form the aluminum salt. Formation of the metal salt of the organic pigment will generally convert the pigment from a water soluble pigment into a water insoluble pigment. Examples of organic pigment families that may be used herein include azo, (including monoazo and diazo), fluoran, xanthene, indigoid, triphenylmethane, anthroquinone, pyrene, pyrazole, quinoline, quinoline, or metallic salts thereof. Preferred are D&C colors, FD&C colors, or Lakes of D&C or FD&C colors. The term “D&C” means drug and cosmetic colors that are approved for use in drugs and cosmetics by the FDA. The term “FD&C” means food, drug, and cosmetic colors which are approved for use in foods, drugs, and cosmetics by the FDA. Certified D&C and FD&C colors are listed in 21 CFR 74.101 et seq. and include the FD&C colors Blue 1, Blue 2, Green 3, Orange B, Citrus Red 2, Red 3, Red 4, Red 40, Yellow 5, Yellow 6, Blue 1, Blue 2; Orange B, Citrus Red 2; and the D&C colors Blue 4, Blue 9, Green 5, Green 6, Green 8, Orange 4, Orange 5, Orange 10, Orange 11, Red 6, Red 7, Red 17, Red 21, Red 22, Red 27, Red 28, Red 30, Red 31, Red 33, Red 34, Red 36, Red 39, Violet 2, Yellow 7, Yellow 8, Yellow 10, Yellow 11, Blue 4, Blue 6, Green 5, Green 6, Green 8, Orange 4, Orange 5, Orange 10, Orange 11, and so on. Suitable Lakes of D&C and FD&C colors are defined in 21 CFR 82.51. Particularly preferred are Lakes formed by the reaction of the organic pigment with a metallic salt such as aluminum, calcium, zirconium, barium, and the like. Suitable reds include pigments from the monoazo, disazo, fluoran, xanthene, or indigoid families or Lakes thereof, such as Red 4, 6, 7, 17, 21, 22, 27, 28, 30, 31, 33, 34, 36, and Red 40. Also suitable are Lakes of such red pigments. Typically the metal salts are aluminum, barium, and the like. Most preferred are Aluminum Lakes of the various red pigments mentioned herein.

Suitable yellows include wherein the yellow pigment is a pyrazole, monoazo, fluoran, xanthene, quinoline, or salt thereof. Suitable yellows include Yellow 5, 6, 7, 8, 10, and 11, as well as Lakes of such yellow pigments. Suitable violets include those from the anthroquinone family, such as Violet 2 and Lakes thereof. Examples of orange pigments are Orange 4, 5, 10, 11, or Lakes thereof.

The organic pigments form the main color component of the invention meaning that the color of the composition is attributable to the organic pigments. While inorganic oxides may be incorporated into the composition, the main color of the lash tint (which is black or brown) is due to the organic pigment. In the preferred embodiment of the invention the lash tint is a rich dark brown or black in color, which is achieved through the use of a combination of organic pigments which are not black or brown in color. The rich deep brown or black color may be achieved by combining organic pigments or Lakes thereof in the red, green, yellow, blue, violet, and orange family. Preferably the lash tint comprises a mixture of red, green, yellow, and blue organic pigments or Lakes thereof and is deep brown or black in color. In the most preferred composition the pigments comprise a mixture of red, green, yellow, and blue organic pigments wherein the pigments are Lakes, namely they are in the form of water insoluble aluminum salts. These preferred compositions may comprise organic pigments in non-Lake form however, since such pigments are water soluble, when used in large amounts such non-Lake organic pigments may be incompatible with the liquid carrier. If non-Lake organic pigments are present, they are generally present at about 0.0001-3%, preferably about 0.0005-1% by weight of the total composition at most. The most preferred compositions of the invention are dark brown or black in color and are free of iron oxides, particularly black iron oxide, or contain such iron oxides in amounts less than about 5-10% by weight.

B. Inorganic Pigments

In the event the claimed composition contains inorganic pigments, preferred is where the amount is sufficient to accentuate the color achieved with the organic pigment but not obscure the intensity of the organic pigments. Preferred ranges include about 0.001-15%, preferably about 0.005-10%, more preferably about 0.01-8% by weight of the total composition. Suitable inorganic pigments include iron oxides such as red, blue, black, green, and yellow; titanium dioxide, bismuth oxychloride, and the like. Preferred are iron oxides.

C. Particulate Fillers

It may also be desirable to include one or more particulate fillers in the claimed composition. If so, suggested ranges are about 0.001-40%, preferably about 0.05-35%, more preferably about 0.1-30% by weight of the total composition. Preferably, the particulate matter has a particle size of 0.02 to 100, preferably 0.5 to 100, microns. Suitable particle fillers include titanated mica, fumed silica, spherical silica, polymethylmethacrylate, micronized teflon, boron nitride, acrylate copolymers, aluminum silicate, aluminum starch octenylsuccinate, bentonite, calcium silicate, cellulose, chalk, corn starch, diatomaceous earth, fuller's earth, glyceryl starch, hectorite, hydrated silica, kaolin, magnesium aluminum silicate, magnesium trisilicate, maltodextrin, montmorillonite, microcrystalline cellulose, rice starch, silk powder, silica, talc, mica, zinc laurate, zinc myristate, zinc rosinate, alumina, attapulgite, calcium carbonate, calcium silicate, dextran, kaolin, nylon, silica silylate, sericite, soy flour, tin oxide, titanium hydroxide, trimagnesium phosphate, walnut shell powder, or mixtures thereof. The above mentioned powders may be surface treated with lecithin, amino acids, mineral oil, silicone oil or various other agents either alone or in combination, which coat the powder surface and render the particles more lipophilic in nature.

II. Liquid Carrier

The composition comprises about 0.1-85%, preferably about 5-80%, more preferably about 10-75% by weight of the total composition of a liquid carrier for the film forming polymer and the organic pigments, which may comprise one or more oils or other liquid materials. Preferably the liquid carrier is anhydrous. The term “anhydrous” means that water is not intentionally added to the composition. A variety of ingredients may be suitable including volatile oils, nonvolatile oils, and mixtures thereof. In the most preferred embodiment of the invention the inorganic pigments are insoluble in the liquid carrier, e.g. they are dispersed rather than solubilized in the liquid carrier.

A. Volatile Liquids

The term “volatile” means that the oil has a measurable vapor pressure, or a vapor pressure of at least 2 mm. of mercury at 20.degree. C. The term “nonvolatile” means that the oil has a vapor pressure of less than 2 mm. of mercury at 20.degree. C. Preferably, the compositions of the invention contain a significant portion of volatile solvents as the liquid carrier. Suitable volatile oils are liquids, and enable easy formulation of the composition of the invention. When the composition of the invention is applied to the desired surface, the volatile solvent of the invention must be capable of flashing off to leave the other ingredients in the composition affixed to the surface. Suitable volatile solvents generally have a viscosity of 0.5 to 10 centipoise at 25.degree. C. Suitable volatile solvents include linear silicones, cyclic silicones, paraffinic hydrocarbons, or mixtures thereof.

B. Nonvolatile Liquids

The liquid carrier may also comprise low viscosity non-volatile liquid oils such as silicones, esters, and the like. If the nonvolatile oils are too heavy or greasy it may hamper the long wearing characteristics of the invention. Generally, the viscosity of the nonvolatile oils if present should range from about 11-1000, preferably less than 100 centipoise, most preferably less than about 50 centipoise at 25.degree. C. Examples of such oils include polyalkylsiloxanes, polyarylsiloxanes, and polyethersiloxanes. Examples of such nonvolatile silicones are disclosed in Cosmetics, Science and Technology 27-104 (Balsam and Sagarin ed. 1972); and U.S. Pat. Nos. 4,202,879 and 5,069,897, both of which are hereby incorporated by references. Further nonlimiting examples of such silicones include dimethicone, phenyl trimethicone, dimethicone copolyol, and so on. Also suitable are lower viscosity organic liquids including saturated or unsaturated, substituted or unsubstituted branched or linear or cyclic organic compounds that are liquid under ambient conditions. Preferred organic liquids include those described in U.S. Pat. Nos. 5,505,937; 5,725,845; 5,019,375; and 6,214,329, all of which are incorporated by reference herein in their entirety. In the preferred composition the liquid carrier comprises one or more nonvolatile liquids either alone or in combination with one or more nonvolatile liquids. Particularly preferred is where the liquid vehicle comprises a mixture of volatile silicone and volatile paraffinic hydrocarbons which serve as the carrier in which the organic pigments are dispersed.

III. Film Forming Polymer

The composition preferably comprises 0.1-35%, preferably 0.5-30%, more preferably 1-25% by weight of the total composition of one or more film forming polymers. The film forming polymer (or film former) may be soluble or dispersible in the liquid carrier and when the composition is applied to the desired surface, the liquid carrier at least partially evaporates and causes the film forming polymer to form a film on the surface which holds the organic pigment particles in place with the network created by the hardened polymer. The term “soluble” means that the film forming polymer is soluble in the liquid vehicle and when combined both components form a homogeneous single phase. The term “dispersable” means that the film forming polymer is readily dispersed in the liquid vehicle and forms a stable, heterogeneous composition where the dispersed polymer remains stable and suspended in the liquid vehicle and is compatible therewith (without settling out, for example). The film forming polymer also has adhesive properties, meaning that when incorporated into the claimed composition and applied to the lashes, the film forming polymer forms a film or a weld on the lashes. Such a film will have adhesive and cohesive strength, as is understood by those skilled in the art. Further, the preferred film forming polymer will be capable of forming an semi-permanent film on the surface to which it is applied, meaning that the composition containing the polymer is not removed from the surface to which it is applied with simple soap and water immediately after application.

A variety of film forming polymers may be suitable so long as they are soluble or dispersible in, and compatible with, the liquid carrier; are capable of forming a film on the lashes that may be removed with a remover; and are compatible with the pigment and the liquid carrier. Such polymers may be natural or synthetic and are further described below.

A. Synthetic Polymers

1. Copolymers of Silicone and Ethylenically Unsaturated Monomers

One type of film forming polymer that may be used in the compositions of the invention is obtained by reacting silicone moieties with ethylenically unsaturated monomers. The resulting copolymers may be graft or block copolymers. The term “graft copolymer” is familiar to one of ordinary skill in polymer science and is used herein to describe the copolymers which result by adding or “grafting” polymeric side chain moieties (i.e. “grafts”) onto another polymeric moiety referred to as the “backbone”. The backbone may have a higher molecular weight than the grafts. Thus, graft copolymers can be described as polymers having pendant polymeric side chains, and which are formed from the “grafting” or incorporation of polymeric side chains onto or into a polymer backbone. The polymer backbone can be a homopolymer or a copolymer. The graft copolymers are derived from a variety of monomer units. One type of polymer that may be used as the film forming polymer is a vinyl-silicone graft or block copolymer.

2. Polymers from Ethylenically Unsaturated Monomers

Also suitable for use as film forming polymers are polymers made by polymerizing one or more ethylenically unsaturated monomers. The final polymer may be a homopolymer, copolymer, terpolymer, or graft or block copolymer, and may contain monomeric units such as acrylic acid, methacrylic acid or their simple esters, styrene, ethylenically unsaturated monomer units such as ethylene, propylene, butylene, etc., vinyl monomers such as vinyl chloride, styrene, and so on. Preferred are polymers containing one or more monomers which are esters of acrylic acid or methacrylic acid, including aliphatic esters of methacrylic acid like those obtained with the esterification of methacrylic acid or acrylic acid with an aliphatic alcohol of 1 to 30, preferably 2 to 20, more preferably 2 to 8 carbon atoms. If desired, the aliphatic alcohol may have one or more hydroxy groups. Also suitable are methacrylic acid or acrylic acid esters esterified with moieties containing alicyclic or bicyclic rings such as cyclohexyl or isobornyl, for example. The ethylenically unsaturated monomer may be mono-, di-, tri-, or polyfunctional as regards the addition-polymerizable ethylenic bonds. A variety of ethylenically unsaturated monomers are suitable.

3. Silicone Polymers

Also suitable are various types of high molecular weight silicone polymers such as silicone gums, resins, and the like.

B. Natural Polymers

Also suitable for use are one or more naturally occurring polymeric materials such as resinous plant extracts including such as rosin, shellac, and the like.

IV. Other Ingredients

A. Plasticizers

It is desirable to incorporate one more plasticizers into the composition. Since the preferred compositions tend to have a lower viscosity when compared to standard mascaras, the plasticizer will improve the spreadability and application of the composition to the surface to which it is applied. The preferred compositions contain one or more plasticizers in an amount sufficient to improve spreadability and application of the composition when compared to the same composition without the plasticizer. Suggested ranges of plasticizers range from about 0.01-20%, preferably about 0.05-15%, more preferably about 0.1-10% by weight of the total-composition. A variety of plasticizers are suitable including Suitable plasticizers include glyceryl, glycol, and citrate esters as disclosed in U.S. Pat. No. 5,066,484, which is hereby incorporated by reference. Examples of such esters include glyceryl tribenzoate, glyceryl triacetate, acetyl tributyl citrate, dipropylene glycol dibenzoate, and the like.

B. Viscosity Modifiers

It may also be desirable to include one or more viscosity modifiers in the composition. In particular, since the preferred compositions exhibit a viscosity that is slightly reduced, inclusion of the viscosity modifiers, which will, specifically increase viscosity, cause the composition to have more body and less tendency to run or drip when applied to the desired surface. Suggested ranges of such viscosity modifiers are about 0.01-60%, preferably about 0.05-50%, more preferably about 0.1-45% by weight of the total composition. Preferred are where the viscosity modifiers are not waxes or wax like materials. Suitable viscosity modifiers include natural or synthetic montmorillonite minerals such as hectorite, bentonite, and quaternized derivatives thereof which are obtained by reacting the minerals with a quaternary ammonium compound, such as stearalkonium bentonite, hectorites, quaternized hectorites such as Quaternium-18 hectorite, attapulgite, carbonates such as propylene carbonate, bentones, and the like. Particularly preferred is Quatemium-18 hectorite.

Also suitable as the viscosity modifier are various polymeric compounds known in the art as associative thickeners. Suitable associative thickeners generally contain a hydrophilic backbone and hydrophobic side groups. Examples of such thickeners include polyacrylates with hydrophobic side groups, cellulose ethers with hydrophobic side groups, polyurethane thickeners. Examples of hydrophobic side groups are long chain alkyl groups such as dodecyl, hexadecyl, or octadecyl; alkylaryl groups such as octylphenyl or nonylphenyl. Another type of viscosity modifier that may be used in the compositions are silicas, silicates, silica silylate, and derivatives thereof. These silicas and silicates are generally found in the particulate form. Particularly preferred is silica.

In the most preferred embodiment of the invention, the compositions are free of wax viscosity modifiers, meaning synthetic and natural waxes such as synthetic wax, castor wax, ceresin, rice wax, and the like, or contain such viscosity modifiers in substantially reduced amounts, generally less than 10% by weight, preferably less than 5% by weight, most preferably less than 1% by weight.

Nail Polish

The nail polish compositions of the present invention includes for example, pigments and dyes that are suspended in a viscous matrix. The viscous matrix typically includes a film forming component and a plasticizer. One type of plasticizer is dibutyl phthalate. The nail polish also includes an adhesion promoter, which may be a polymeric component formed by the condensation polymerization of for example formaldehyde or other sulfonamide formaldehyde resin.

We claim:

1. A cosmetic composition wherein there is an additive, said additive comprising one or more hydrogels made from a poly-γ-glutamic acid (pga) compound.

2. A cosmetic composition wherein there is an additive, said additive comprising a hyaluronic acid (HA).

3. A cosmetic composition wherein there is an additive, said additive comprising a chitosan based composition.

4. The additive according to claim 1, 2 or 3 wherein the additive is used in a lip product.

5. The additive according to claim 1, 2 or 3 wherein the additive is used in a lip stick

6. The additive according to claim 1, 2 or 3 wherein the additive is used in a pomade

7. The additive according to claim 1, 2 or 3 wherein the additive is used in a lip balm.

8. The additive according to claim 1, 2 or 3 wherein the additive is used in a skin cream.

9. The additive according to claim 1, 2 or 3 wherein the additive is used in mascara.

10. The additive according to claim 1, 2 or 3 wherein the additive is used in a nail polish.

11. The additive according to claim 1, 2 or 3 wherein the additive is used in a lotion.

12. The additive according to claim 1, 2 or 3 wherein the additive is used in a shampoo.

13. The additive according to claim 1, 2 or 3 wherein the additive is used in a hair conditioner.

14. The additive according to claim 1, 2 or 3 wherein the additive is used in a hair dye.

15. The additive according to claim 1, 2 or 3 wherein the additive is used in a sunscreen.

16. The additive according to claim 1, 2 or 3 wherein the additive is used in a deodorant.

17. The additive according to claim 1, 2 or 3 wherein the additive is used in an anti aging cream.

18. The additive according to claim 1, 2 or 3 wherein the additive is used in skin care product.
